(19) United States
(12) Patent Application Publication
(10) Pub. No.: US 2010/0063547 A1
(43) Pub. Date: Mar. 11, 2010

(54) DYNAMIC MOTION SPINAL STABILIZATION SYSTEM AND DEVICE

(76) Inventors: Joshua Morin, Newington, CT (US); Arnold Oyola, Northborough, MA (US); Wayne Boucher, Manchester, NH (US)

Correspondence Address: MIDDLETON & REUTLINGER, 2500 BROWN & WILLIAMSON TOWER, LOUISVILLE, KY, 40202, US

(21) Appl. No.: 12/435,231

(22) Filed: May 4, 2009

Related U.S. Application Data
(60) Provisional application No. 61/050,082, filed on May 2, 2008.

(51) Int. Cl. A61B 17/70

(52) U.S. Cl. 606/278

(57) ABSTRACT

A dynamic motion component for a spinal implant is provided, comprising a first rod coupled to an enclosure and a second end of a second rod captured in a cavity of the enclosure. A dampener unit surrounds a captured portion of the second end and is positioned between the first end and the second end. In response to pivotal or translational movement of the second rod relative to the first rod, the dampener unit is compressed against one or more inner surfaces of the cavity to provide for progressive resistance of movement of the second rod.

### RELATED APPLICATIONS

This application relates to, and claims the benefit of the filing date of, co-pending U.S. provisional patent application Ser. No. 61/050,082 entitled “Dynamic Motion Spinal Stabilization System and Device”, filed May 2, 2008, the entire contents of which are incorporated herein by reference for all purposes. This application is related to U.S. Provisional Patent Application 61/031,645, entitled “Dynamic Spinal Implants and Method of Use,” filed on Feb. 26, 2008; U.S. patent application Ser. No. 11/738,990, entitled “Dynamic Motion Spinal Stabilization System and Device,” filed on Apr. 23, 2007; U.S. patent application Ser. No. 11/693,394, entitled “Dynamic Motion Spinal Stabilization System,” filed on Mar. 29, 2007; U.S. Provisional Patent Application 60/863,284, entitled “Alignment Instrument for Dynamic Spinal Stabilization Systems,” filed on Oct. 27, 2006; U.S. Provisional Patent Application 60/826,763, entitled “Alignment Instrument for Dynamic Spinal Stabilization Systems,” filed on Sep. 25, 2006; U.S. Provisional Patent Application 60/825,078, entitled “Offset Adjustable Dynamic Stabilization System,” filed on Sep. 8, 2006; U.S. patent application Ser. No. 11/467,798, entitled “Alignment Instrument for Dynamic Spinal Stabilization Systems,” filed on Aug. 28, 2006; U.S. Provisional Patent Application 60/831,879, entitled “Locking Assembly,” filed on Jul. 19, 2006; U.S. Provisional Patent Application 60/793,829, entitled “Micro Motion Spherical Linkage Implant System,” filed on Apr. 21, 2006; U.S. patent application Ser. No. 11/303,138, entitled “Three Column Support Dynamic Stabilization System and Method,” filed on Dec. 16, 2005; and U.S. patent application Ser. No. 10/914,751, entitled “System and Method for Dynamic Skeletal Stabilization,” filed on Aug. 9, 2004; All of the above applications are incorporated by reference herein in their entirety for all purposes.

### FIELD OF THE INVENTION

This disclosure relates to skeletal stabilization and, more particularly, to systems and method for stabilization of human spines and, even more particularly, to dynamic stabilization techniques.

### BACKGROUND

The human spine is a complex structure designed to achieve a myriad of tasks, many of them of a complex kinematic nature. The spinal vertebrae allow the spine to flex in three axes of movement relative to the portion of the spine in motion. These axes include the horizontal (bending either forward/anterior or aft/posterior), roll (bending to either left or right side) and vertical (twisting of the shoulders relative to the pelvis).

In flexing about the horizontal axis into flexion (bending forward or anterior) and extension (bending backward or posterior), vertebrae of the spine must rotate about the horizontal axis to various degrees of rotation. The sum of all such movement about the horizontal axis of produces the overall flexion or extension of the spine. For example, the vertebrae that make up the lumbar region of the human spine move through roughly an arc of 15° relative to its adjacent or neighboring vertebrae. Vertebrae of other regions of the human spine (e.g., the thoracic and cervical regions) have different ranges of movement. Thus, if one were to view the posterior edge of a healthy vertebrae, one would observe that the edge moves through an arc of some degree (e.g., of about 15° in flexion and about 5° in extension if in the lumbar region) centered about a center of rotation. During such rotation, the anterior (front) edges of neighboring vertebrae move closer together, while the posterior edges move farther apart, compressing the anterior of the spine. Similarly, during extension, the posterior edges of neighboring vertebrae move closer together while the anterior edges move farther apart thereby compressing the posterior of the spine. During flexion and extension the vertebrae move in horizontal relationship to each other providing up to 2-3 mm of translation.

In a healthy spine the inter-vertebral spacing between neighboring vertebrae is maintained by a compressible and somewhat elastic disc. The disc serves to allow the spine to move about the various axes of rotation and through the various arcs and movements required for normal mobility. The elasticity of the disc maintains spacing between the vertebrae during flexion and lateral bending of the spine thereby allowing room or clearance for compression of neighboring vertebrae. In addition, the disc allows relative rotation about the vertical axis of neighboring vertebrae allowing twisting of the shoulders relative to the hips and pelvis. A healthy disc further maintains clearance between neighboring vertebrae thereby enabling nerves from the spinal chord to extend out of the spine between neighboring vertebrae without being squeezed or impinged by the vertebrae.

In situations where a disc is not functioning properly, the inter-vertebral disc tends to compress thereby reducing inter-vertebral spacing and exerting pressure on nerves extending from the spinal cord. Various other types of nerve problems may be experienced in the spine, such as exiting nerve root compression in the neural foramen, passing nerve root compression, and enervated annulus (where nerves grow into a cracked/compromised annulus, causing pain every time the disc/annulus is compressed), as examples. Many medical procedures have been devised to alleviate such nerve compression and the pain that results from nerve pressure. Many of these procedures revolve around attempts to prevent the vertebrae from moving too close to each in order to maintain space for the nerves to exit without being impinged upon by movements of the spine.

In one such procedure, screws are embedded in adjacent vertebrae pedicles and rigid rods or plates are then secured between the screws. In such a situation, the pedicle screws press against the rigid spacer which serves to distract the degenerated disc space thereby maintaining adequate separation between the neighboring vertebrae to prevent the vertebrae from compressing the nerves. Although the foregoing procedure prevents nerve pressure due to extension of the spine, when the patient then tries to bend forward (putting the spine in flexion), the posterior portions of at least two vertebrae are effectively held together. Furthermore, the lateral bending or rotational movement between the affected vertebrae is significantly reduced, due to the rigid connection of the spacers. Overall movement of the spine is reduced as more vertebras are distracted by such rigid spacers. This type of spacer not only limits the patient's movements, but also places additional stress on other portions of the spine, such as adjacent vertebrae without spacers, often leading to further complications at a later date.

Accordingly, dynamic systems which approximate and enable a fuller range of motion while providing stabilization of a spine are needed.

### SUMMARY OF INVENTION

A dynamic motion component for a spinal implant is provided, comprising a first rod extending from an enclosure having a cavity, and a second end of a second rod captured in the cavity. A dampener unit surrounds a captured portion of the second end and is positioned between the first rod and the second end of the second rod, within the enclosure. In response to pivotal or translational movement of the second rod relative to the first rod, the dampener unit is compressed against one or more inner surfaces of the cavity to provide for progressive resistance against movement of the second rod.

### BRIEF DESCRIPTION OF THE DRAWINGS

For a more complete understanding of the present invention and the advantages thereof, reference is now made to the following Detailed Description taken in conjunction with the accompanying drawings, in which:

FIG. 1 is an isometric view of an embodiment of a dynamic stabilization system coupled to a pair of adjacent vertebrae.

FIG. 2 is an exploded view of one possible embodiment of a dynamic stabilization brace which may be incorporated in the dynamic stabilization system of FIG. 1.

FIG. 3 is a cross sectional view of one possible embodiment of a dampener which may be incorporated in the dynamic brace of FIG. 2.

FIG. 4 is a cross section view of one possible embodiment of a closure member which may be incorporated in the dynamic brace of FIG. 2.

FIG. 5 is a cross sectional view of the dynamic stabilization brace of FIG. 2.

FIG. 6A is a cross sectional view of the dynamic stabilization brace of FIG. 2 in a first possible position.

FIG. 6B is a cross sectional view of the dynamic stabilization brace of FIG. 2 in a second possible position.

### DETAILED DESCRIPTION

It is to be understood that the following disclosure provides many different embodiments, or examples, for implementing different features of the disclosure. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

Certain aspects of the present disclosure provide dynamic stabilization systems, dynamic stabilization devices, and/or methods for maintaining spacing between consecutive neighboring vertebrae and stabilizing a spine, while allowing movement of the vertebrae relative to each other. The neighboring vertebrae may be immediately next to each other or spaced from each other by one or more intervening vertebrae.

It is sometimes difficult to match a dynamic stabilization system with a particular patient's anatomical structure while ensuring that a minimum range of motion is available for the dynamic implant due to factors such as the variability of pedicle to pedicle distance in the lumbar spine.

Accordingly, the following disclosure describes dynamic stabilization systems, devices, and methods for dynamic stabilization which may provide for adjustable distraction of the inter-vertebral space while still allowing a patient a substantial range of motion in two and/or three dimensions. Such a dynamic stabilization system may allow the vertebrae to which it is attached to move through a natural arc that may resemble an imaginary three dimensional surface such as a sphere or an ellipsoid. Accordingly, such a system may aid in permitting a substantial range of motion in flexion, extension, and/or other desired types of natural spinal motion.

Although only a few exemplary embodiments of this disclosure have been described in details above, those skilled in the art will readily appreciate that many modifications are possible in the exemplary embodiments without materially departing from the novel teachings and advantages of this disclosure. Also, features illustrated and discussed above with respect to some embodiments can be combined with features illustrated and discussed above with respect to other embodiments. Accordingly, all such modifications are intended to be included within the scope of this disclosure.

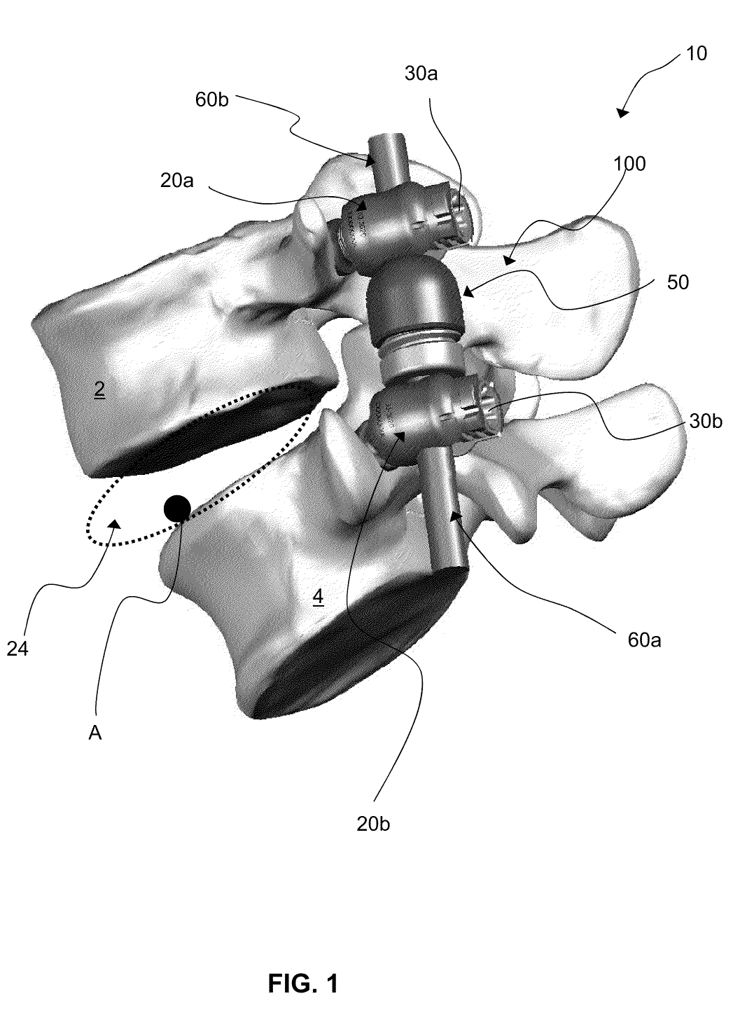

Referring to FIG. 1, there is illustrated one possible embodiment of a dynamic stabilization system 10 which may be used to dynamically stabilize one or more bony structures, such as a pair of adjacent vertebrae 2 and 4. The dynamic stabilization system 10 may include a pair of bone anchors 20a and 20b anchored to one or more vertebrae 2 and 4 and a dynamic brace 100 coupled between the pair of bone anchors 20a and 20b.

In some embodiments, the relative movement of the dynamic brace 100 may be limited to a path having a central point “A” (e.g., a center of rotation) within an intervertebral disc space 24. The point “A” may be stationary or may move within the space 24 in conjunction with movement of the vertebrae to which the dynamic brace 100 is coupled. Furthermore, the point “A” need not be a stationary point, but may follow a path on or through the space 24. For purposes of convenience, the term center of rotation (“COR”) may be used herein to refer to a specific point and/or a three dimensional area.

The dynamic brace 100 may include a dynamic motion component 50 coupled to a pair of elongated members 60a and 60b, which may couple to the respective bone anchors 20a and 20b. In some embodiments, the dynamic motion component may comprise an enclosure for housing ends of the pair of elongated members 60a and 60b. As will be explained in greater detail below, the dynamic motion component 50 may allow the pair of elongated members 60a and 60b to move in a controlled manner in respect to one another. Each bone anchor 20a and 20b may have a head with a channel that is dimensioned to receive the pair of elongated members 60a and 60b. A pair of locking members 30a and 30b may be used to secure the elongated members 60a and 60b within the channel of the respective bone anchors 20a and 20b. The locking members 30a and 30b may threadingly couple to the head of the bone anchors 20a and 20b and may compress or lock against the respective elongated members 60a and 60b. Any embodiment of a dynamic brace described herein may be coupled to a pair of bone anchors in a similar fashion. The bone anchors 20a and 20b may be monoaxial or polyaxial pedicle screws having a swivel head (as shown). The bone anchors 20a and 20b may also include hooks, plates or other anchors known to those skilled in the art.

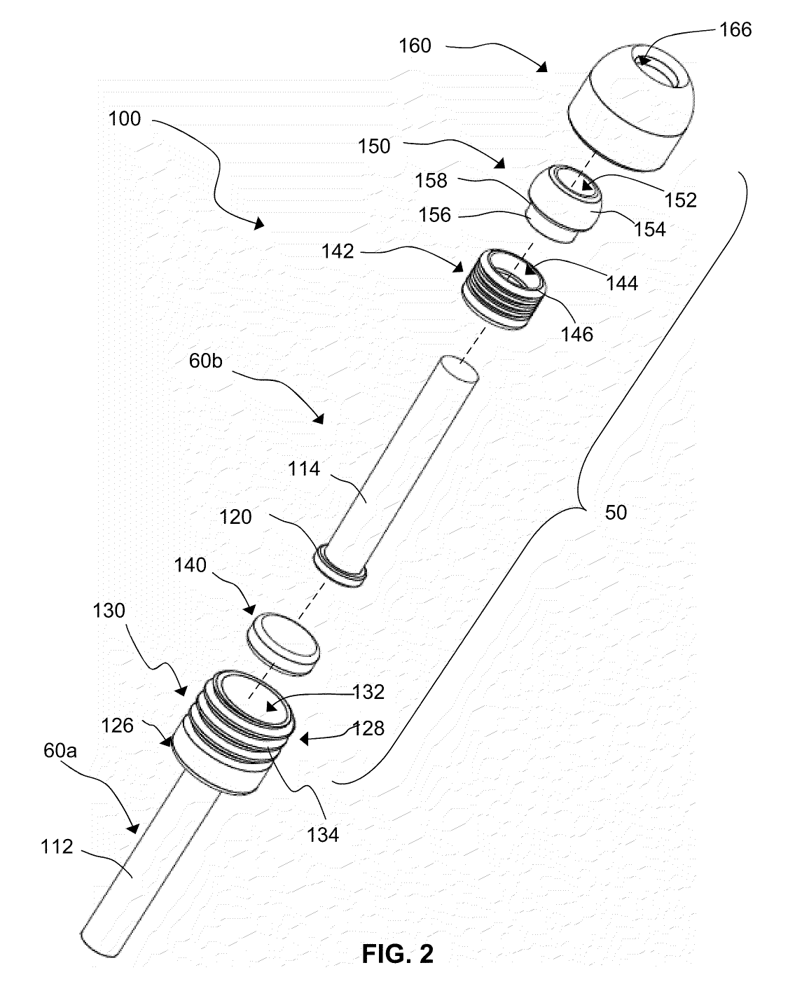

Referring to FIG. 2, an exploded assembly view of the dynamic brace 100 of FIG. 1 is shown illustrating the dynamic motion component 50 extending generally along a longitudinal axis 10. The dynamic motion component 50 may include a second enlarged end portion 120 of the second elongated member 60b, a housing 130, a closure member 160, a spherical bushing 150, and a pair of dampeners 140 and 142. The dynamic motion component 50 may allow the elongated members 60a and 60b to rotate, pivot and translate relative to one another after the elongated members 60a and 60b have been rigidly secured to the heads 20a and 20b (respectively), as shown in FIG. 1. The elongated members 60a and 60b may have a compound motion in which rotation, pivoting and translation occur at the same time. The rotational, pivoting and translation movements of the elongated members 60a and 60b may be independent of one another, for example translation may occur with or without any rotational or pivoting motion. The dynamic motion component 50 may control or limit the COR within a specific defined boundary. When the dynamic brace 100 is coupled to the pair of vertebrae 2 and 4, as shown in FIG. 1, the dynamic brace 100 may allow the pair of vertebrae 2 and 4 to move in flexion, extension and lateral bend.

The housing 130 may have a generally cylindrical shape with a proximal end portion 126 and a distal end portion 128 having an inner surface defining a recess 132. The recess 132 may be dimensioned to receive the first dampener 140. An external surface 134 of the housing 130 may be threaded to aid in assembly of the dynamic brace 100. The rod portion 112 of the first elongated member 60a and the housing 130 may be machined as one piece or the first elongated member 60a may be fixed to the housing 130 by welding, pinning, press fitting or other commonly used assembly methods. The housing 130 and the first elongated member 60a may be manufactured from metals such as titanium, stainless steel or cobalt chrome. Alternatively the housing 130 and the first elongated member 60a may be manufactured from high strength polymers such as PEEK (poly ether ether ketone). The first elongated member 60a may be manufactured from the same material as the housing 130 or a different material.

The second elongated 60b member may be manufactured from similar materials as the first elongated member 60a. The second elongated member 60b may include the second enlarged end portion 120 and a second rod portion 114. The second enlarged end portion 120 may be sized to fit within the recess 132 of the housing 130. The second rod portion 114 may be sized to fit through a first bore 144 of the second dampener 142, a second bore 152 of the bushing 150 and a third bore 166 of the closure member 160. The second dampener 142, the bushing 150 and the closure member 160 will be described in greater detail below.

The bushing 150 may have an inner surface defining a second bore 152 extending there through that is dimensioned to receive the second rod portion 114 of the second elongated member 60b. The bushing 150 may have a first spherical end portion 154 and a second end portion 156 having a shoulder 158. The second end portion 156 of the bushing 150 may be positioned within the first bore 144 of the second dampener 142 such that the shoulder 158 is positioned against a first end surface 146 of the second dampener 142.

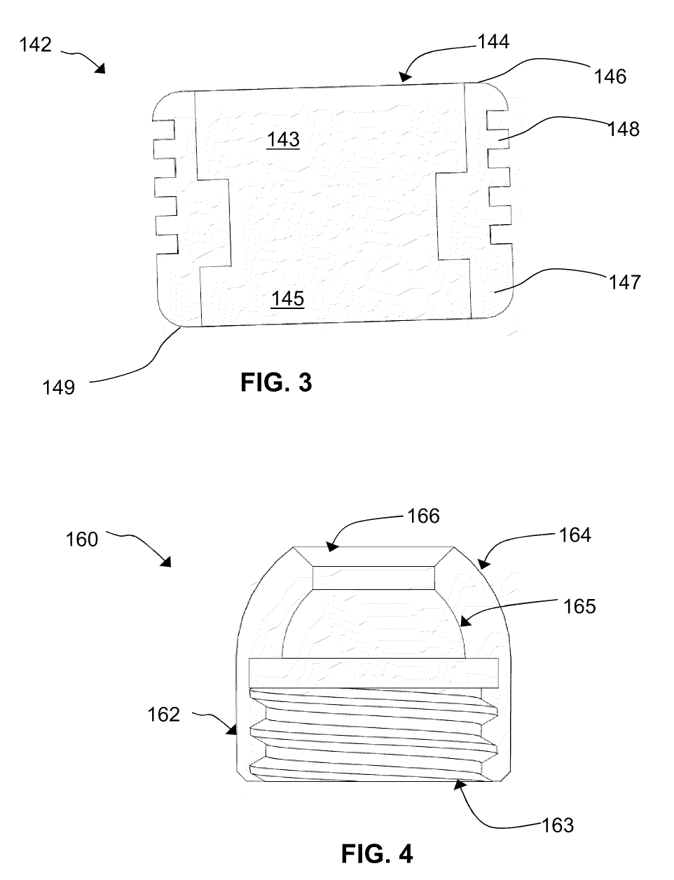

Referring now to FIG. 3, a cross sectional view of the second dampener 142 is shown. The second dampener 142 may be generally cylindrical in shape with the inner surface defining the first bore 144 which may extend completely through the second dampener 142. The inner surface may also define a first recessed portion 143 having a first shoulder and a second recessed portion 145 having a second shoulder. The first recessed portion 143 may be dimensioned to receive the second end portion 156 of the bushing 150 (not shown), as previously described. The second recessed portion 145 of the second dampener 142 may be dimensioned to receive the second enlarged end portion 120 of the second elongated member 60b (not shown). The second dampener 142 may have side walls having thick wall sections 147 and ribs 148 which may allow for thinner wall sections. As will be described in greater detail below, the thick wall sections 147 and ribs 148 may allow for varying stiffness along a length of the second dampener 142 which may aid in controlling various motions of the dynamic brace 100.

FIG. 4 illustrates a cross section view of one embodiment of the closure member 160 which may mate with the housing 130 (not shown). The closure member 160 may have a generally cylindrical shaped first end portion 162 and a generally spherical shaped second end portion 164. The first end portion 162 may have an inner surface that defines a threaded bore 163. The inner surface may be dimensioned to at least partially receive the housing 130 (not shown) and the threaded bore 163 may mate with the external threads 134 of the housing 130 (not shown). The second end portion 164 may have an end wall that defines an opening 166. The second end portion 164 may have a spherical inner surface 165 that is in communication with the opening 166 and the threaded bore 163. The spherical inner surface 165 may be dimensioned to receive the spherical portion 154 of the bushing 150 (not shown). The closure member 160 may be manufactured from metals such as titanium, stainless steel or cobalt chrome. Alternatively the closure member may also be manufactured from high strength polymers such as PEEK (poly ether ether ketone).

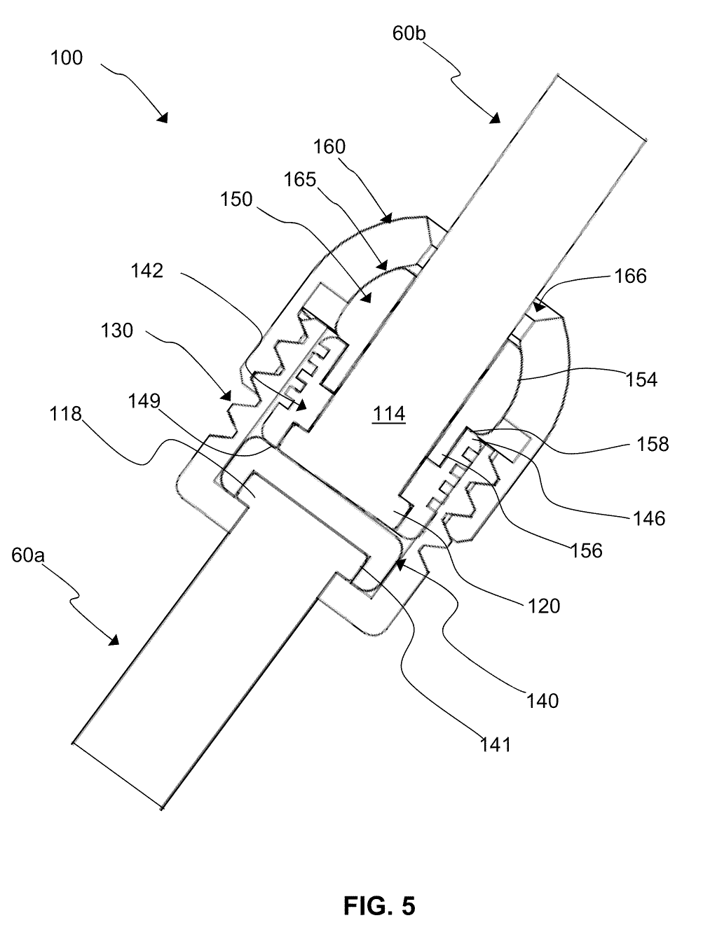

Referring now to FIG. 5, a cross sectional view of the dynamic brace 100 is shown. The first elongated member 60a may have a first enlarged end 118 that is positioned at a distal end portion of the recess 132 of the housing 130. The first dampener 140 may be substantially disc shaped with a first end portion defining a recess 141. The first dampener 140 may be positioned within the housing 130 such that the first enlarged end portion 118 is positioned within the recess 141. The first and second dampeners 140 and 142 may be injection molded or machined from polymers or elastomers, such as Bionate® polycarbonate-urethane (hardness grade 55D) from Polymer Technology Group, Inc. (2810 7th St. Berkeley, Calif. 94710). The first and second dampeners 140 and 142 may also include various types of spring elements such as extension springs, compression springs and wave springs.

The second dampener 142 may be positioned within the housing 130 such that a second end surface 149 of the second dampener 142 may be adjacent to or contacting the first dampener 140 along the longitudinal axis 10. The second rod portion 114 may be positioned within the first bore 144 (see FIG. 3) of the second dampener 142. The second enlarged end portion 120 may be positioned adjacent to the first dampener 140 and within the recess 145 (see FIG. 3) of the second dampener 142. The first recessed portion 143 (see FIG. 3) of the second dampener 142 may receive the second end portion 156 of the bushing 150 such that the shoulder 158 is positioned adjacent or against the first end surface 146 of the second dampener 142. The second rod portion 114 may be positioned within second bore 152 (See FIG. 2) of the bushing 150.

The closure member 160 may threadingly couple to the housing 130 to form a cavity in the enclosure formed by the closure member 160 and the housing 130 to capture the first and second dampeners 140 and 142, the bushing 150 and the second elongated member 60b within the housing 130. As will be explained in greater detail below, alternative embodiments may include the housing 130 being adjustably fixed to the closure member 132, which may allow a surgeon to adjust a compression force of the dampeners 140 and 142, and thus enhance or restrict the level of motion of the dynamic brace 100. Other assembly methods may be used in addition to the threads to fix the position of the housing 130 relative to the closure member 132, such as set screws, press fit pins, welding, adhesives and locking washers. The second rod portion 114 may extend through the third bore 166 of the closure member 160. The third bore 166 of the closure member 160 may be sized to allow the second rod portion 114 to pivot and rotate within the housing 130 and the closure member 160. The first spherical end portion 154 of the bushing 150 may bear against the spherical inner surface 165 (see FIG. 4) of the closure member 160 as the bushing pivots and rotates with respect to the closure member 160. The spherical bushing 150 may control or prescribe the motion of the dynamic brace 100 The bushing 150 and the spherical inner surface 165 of the closure member 160 (see FIG. 4) may be manufactured from materials with superior bearing properties and wear resistance. For example, the bushing 150 may be machined or molded from PEEK and the spherical inner surface 165 of the closure member 160 may be cobalt chrome.

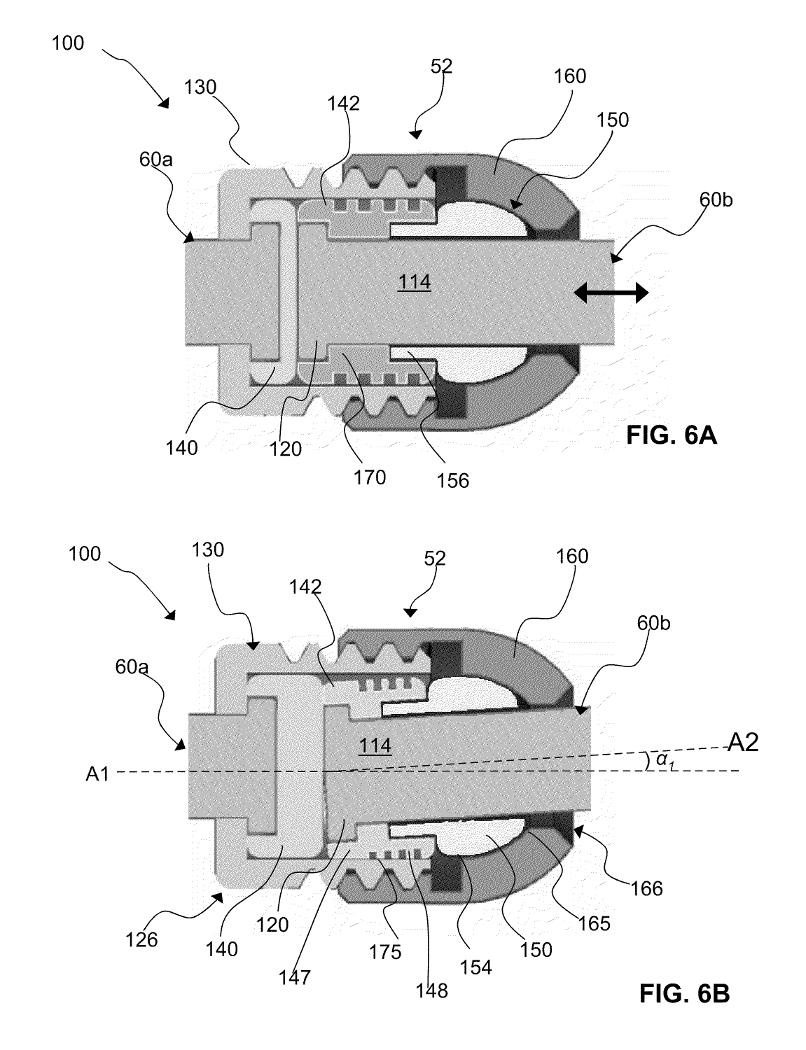

To control and allow various movements of the spine such as flexion, extension and lateral bending the dynamic brace 100 may need to pivot, translate and rotate independently and/or simultaneously. Referring to FIG. 6A a detailed cross sectional view of the dynamic brace 100 is shown in a possible first position. The first position may represent a position of the dynamic brace 100 coupled to a pair of vertebrae of a spine that is in extension. The extension of the spine may result in the second enlarged portion 120 of the second elongated member 60b translating or sliding within the second dampener 142 and towards the first elongated member 60a. The second rod portion 114 may also slide or translate within the bushing 150 and the second dampener 142. The second enlarged portion 120 may compress directly or indirectly against the first dampener 140, which may provide for progressive resistance or breaking as the second elongated member 60b reaches a first translational or positional limit. The first dampener 140 may act as a soft stop, bumper, dampener, or cushion to prevent further translation of the second elongated member 60b against the first elongated member 60a and/or the housing 130. The progressive breaking and soft stop may reduce harmful impact to spinal anatomy and the vertebrae to which the dynamic brace 100 is coupled. The dynamic brace 100 with progressive breaking and soft stops may better mimic the function of a human anatomy which is not rigid, but flexible. In certain surgical procedures a majority of spinal anatomy may need to be removed in order to insert a dynamic fixation device or system. This anatomy previously acted as a cushion to slow down or control the forces acting on the spine during movements such as flexion, extension or lateral bend. After this anatomy is removed the importance of providing improved controlled motion through the use of progressive breaking or soft stops increases in order to augment the remaining spinal anatomy.

The first dampener 140 and the second dampener 142 may act as at least a portion of a dampener unit for controlling relative motion of the first elongated member 60a and the second elongated member 60b so that the translation of the second elongated member 60b may be controlled in one direction by the first dampener 140 (as previously described) and in another direction by the second dampener 142. As the second enlarged portion 120 translates or moves axially away from the first dampener 140, the second enlarged portion 120 may compress against a first shoulder 170 of the second dampener 142. The shoulder 170 may be compressed between the second enlarged end portion 120 and second end portion 156 the bushing 150. The compression of the first shoulder 170 of the second dampener 142 may prevent the bushing 150 from being pressed too tightly against the spherical inner surface 165 of the closure member 160. If the bushing 150 is pressed too much against the closure member 160, motion of the dynamic brace 100 may be reduced or excess wear may occur between the bushing 150 and the closure member 160, which may lead to debris particles. The second dampener 142 may act as a second soft stop which allows for gradual cushioning or breaking of the dynamic brace 100 as the second elongated member 60b translates in relation to the first elongated member 60a and reaches a second translational or positional limit in which further translation is prevented.

In certain embodiments the first and second dampeners 140 and 142 may work in conjunction with each other to constantly exert a force against the second elongated member 60b. As the first dampener 140 relaxes, the second dampener 142 may be become compressed, which may result in a force constantly acting on the second elongated member 60b and thus the dynamic brace 100. In this particular embodiment the first and second dampeners 140 and 142 may not allow for unconstrained translation the second elongated member 60b.

Referring to FIG. 6B the dynamic brace 100 is shown in a second possible position, which may represent a position of the dynamic brace 100 when the vertebrae of the spine are in flexion. In the second position the second elongated member 60b may translate, pivot and/or rotate within the housing 130 and in relation to the first elongated member 60a. The translational motion of the second elongated member 60b may be controlled at least in part by the first and second dampeners 140 and 142, as described above.

The second elongated member 60b, second dampener 142 and bushing 150 may be coupled to one another to act as at least a portion of the dampener unit for controlling motion of the second elongated member 60b and the first elongated member 60a such that they pivot together about an axis A1 of the first elongated member 60a. As the second elongated member 60b pivots within the housing 130 and the closure member 160, the first spherical end portion 154 may slide and pivot against the spherical inner surface 165 of the closure member 160. The pivoting motion of the second elongated member 60b may in turn cause the second dampener 142 to pivot and compress against an inner wall 175 of the housing 130.

The second elongated member 60b, second dampener 142 and bushing 150 may act as a unit and pivot or tilt relative to axis A1 resulting in angle (α₁). In certain embodiments the angle (α₁) may be limited to a range of one to five degrees and preferably within a range of three to four degrees. The first elongated member 60a may pivot or tilt in any direction about axis A1, which may allow for the dynamic brace 100 to be coupled to the bone anchors 20a and 20b in any orientation, as shown in FIG. 1.

The pivoting of second elongated member 60b, second dampener 142 and bushing 150 may be limited in several possible ways. The second dampener 142 may provide for cushioning and progressive breaking of the second elongated member 60b until the second dampener 142 reaches its compression limit. The compression limit of the second dampener 142 may act as soft stop to prevent further pivoting of the second dampener 142 against the housing 130.

The opening 166 of the closure member 160 may be dimensioned to allow the second rod portion 114 to pivot 360 degrees in a generally sweeping conical fashion without contacting the closure member 160. The opening 166 of the closure member 160 may allow for a gap between the second rod portion 114 and the closure member 160, which may prevent the closure member 160 from acting as a hard stop and thus allow the second dampener 142 and the housing 130 to act as a soft stop for progressive breaking under normal physiological loads. At forces or loads above normal physiological conditions the opening 166 may be dimensioned such that the closure member 160 contacts the second rod portion 114 to restrict further motion or hyper-mobility. The second enlarged segment 120 may pivot against the first dampener 140, which may provide additional cushioning.

The second elongated member 60b may be free to rotate within the bushing 150 and the second dampener 142. The second elongated member 60b may rotate about its own central longitudinal axis A2, as shown in FIG. 6B. The ability of the second elongated member 60b, the bushing 150 and the second dampener 142 to move and/or rotate independently of each other and the housing 130 may aid the placement and positioning the dynamic brace 100 as well as allow for increase motion of the dynamic brace 100. Alternatively, the second elongated member 60b, the second dampener and the bushing 150 may rotate as a unit within the housing 130. The first spherical end portion 154 and the spherical inner surface 165 may allow for smooth controlled rotation of the second elongated member 60b about axis A2. The smooth and controlled rotation and pivoting (as previously described) may be enhanced by the first spherical end portion 154 remaining in constant contact with the spherical inner surface 165 of the closure member 160 during pivoting and rotation of the second elongated member 60b.

In certain embodiments, the position of the closure member 160 relative to the housing 130 may be adjusted to stiffen movement of the various components (the first and second dampeners 140 and 142, the bushing 150, and the first and second elongated member 60a and 60b) within the housing 130. As the closure member 160 is tightened, the first and second dampeners 140 and 142 may become more compressed within the housing 130, which may result in a stiffer dynamic component 50. If the stiffness of the dynamic component 50 is increased, the pivoting, translational and rotational movements of the dynamic brace 100 may be restricted or limited. The closure member 160 may compress the first and second dampeners 140 and 142 to such a degree that little or no movement (micromotion) of the second elongated member 60b (and thus the dynamic brace 100) is permitted. The COR thus may be restricted to a smaller area by adjusting the amount the first and second dampeners 140 and 142 are compressed.

The motion or movement of the second elongated member 60b may also be varied by increasing or decreasing the thickness or hardness of the first and second dampeners 140 and 142. The thickness of the first and second dampeners 140 and 142 may be varied in specific regions to further control the motion of the second elongated member 60b. For example ribs 148 may be positioned towards the closure member 160 and the thick wall sections 147 may be positioned towards the proximal end portion 126 of the housing 130. The positioning of the ribs 147 may allow for increased motion (such as pivoting of the second elongated member) as the second elongated member 60b translates away from the first elongated member 60a. As the second elongated member 60b translates closer to the first elongated member 60a, motion (such as pivoting of the second elongated member 60b may be restricted by the positioning of the thick wall section 147 of the second dampener 142. In other embodiments the positioning of the thick sections 147 and the ribs 148 may be reversed to permit less motion as the second elongated member 60b translates further from the first elongated member 60a and more motion as the second elongated member 60b translates closer to the first elongated member 60a. It is understood that the first dampener 140 may also have wall sections of varying thickness as described for the second dampener 142 to aid in controlling the motion of the dynamic brace 100.

It is understood that other positions are also possible which may include varying degrees and combinations of translation, pivoting and/or rotation of the second elongated member 60b relative to the first elongated member 60a. These movements may result in varying positions of the second elongated member within the housing 130 and varying amounts of compression on the first and second dampeners 140 and 142. In one preferred embodiment the second elongated member 60b may be cushioned within the housing throughout any and all movements of the second elongated member 60b.

What is claimed is:

1. A dynamic motion component system for controlling spinal movement, the system comprising:
a first rod member coupled to an enclosure at a first rod end, wherein the first rod member extends from the enclosure in generally a first direction;
a second rod member having a second end, wherein the second rod end is captured within a cavity in the enclosure and the second rod member extends out from the enclosure through an opening in generally a second direction opposite from the first direction;
a dampener unit surrounding a captured portion of the second end and positioned within the enclosure between the first rod member and the second rod member;
wherein, in response to pivotal or translational movement of the second rod member relative to the first rod member, the second end of the second rod member compresses one or more portions of the dampener unit against one or more inner surfaces of the cavity to provide progressive resistance to movement of the second rod member.

2. A dynamic motion component system for controlling spinal movement, the system comprising:
a first spring member positioned between a first end of a first rod member and a second end of a second rod member, wherein the first spring member, the first end, and the second end are configured to extend within an enclosure along a longitudinal axis of the enclosure in at least a first position;
a second spring member adjacent to the first spring member positioned on an opposite side from the first end of the first rod member and extending along the longitudinal axis, wherein the second spring member comprises one or more first shoulders positioned between the second end of the second rod member and a first inner surface of the enclosure;
wherein, in response to longitudinal relative movement of the second end of the second rod member towards the first end of the first rod member, the second end is positioned to compress at least a portion of the first spring member against the first end to provide progressive resistance to the movement of the second rod member towards the first rod member; and
wherein, in response to longitudinal relative movement of the second end of the second rod member away from the first end of the first rod member, the second end is positioned to compress against the first shoulder of the second spring member and against the first inner surface of the enclosure to provide progressive resistance to the movement of the second rod member away from the first rod member.

3. The system of claim 2, further comprising:
a first bushing coupled to the second spring member for pivotal movement with the second spring member and positioned between the first shoulder of the second spring member and the first inner surface of the enclosure, wherein the first bushing comprises at least a first outer surface configured to make contact with the first inner surface of the enclosure; and
wherein, in response to pivotal movement of the second rod member relative to the longitudinal axis, the first outer surface of the first bushing pivots with the second rod member to make contact against the first inner surface of the enclosure and the second spring member is compressed by the second rod member against one or more second inner surfaces of the enclosure to provide progressive braking of the pivoting of the second rod member.

4. A spinal dynamic implant for controlling spinal movement, the implant comprising:
an enclosure having a cavity for coupling a first end of a first rod member and a second end of a second rod member, wherein the first rod member and the second rod member extend away in substantially opposite directions from the enclosure and are configured to couple to one or more bone anchors;
a first dampener positioned between the first end and the second end, wherein the first dampener, the first end, and second end are configured to extend within the enclosure along a longitudinal axis of the enclosure in at least a first position;
a second dampener surrounding the second end and positioned adjacent to the first dampener on a side opposite from the first end of the first rod member and extending along the longitudinal axis, wherein the second dampener comprises one or more first shoulders positioned between the second end of the second rod member and a first inner surface of the enclosure;
a first bushing surrounding the second end positioned adjacent to the second dampener between the second dampener and the first inner surface of the enclosure, wherein the first bushing comprises at least a first outer surface configured to make contact with the first inner surface of the enclosure;
wherein, in response to linear translation of the second rod member towards the first rod member, the first dampener is compressed to provide a first soft stop;
wherein, in response to linear translation of the second rod member away from the first rod member, the second dampener and the first bushing are compressed against the first inner surface of the enclosure to provide a second soft stop; and
wherein, in response to pivotal movement of the second rod member relative to the longitudinal axis, the first outer surface of the first bushing pivots with the second rod member to make contact against the first inner surface of the enclosure and the second dampener is compressed against one or more second inner surfaces of the enclosure to provide a third soft stop to provide progressive braking of the pivoting of the second rod member.
